(19) United States
(12) Patent Application Publication
(10) Pub. No.: US 2024/0230633 A1
(43) Pub. Date: Jul. 11, 2024

(54) ENZYME-CONJUGATED MAGNETIC BEADS SUSPENDED IN INTERNAL STANDARD BUFFER

(71) Applicant: DH Technologies Development Pte. Ltd., Singapore (SG)

(72) Inventors: Aaron STELLA, Derry, NH (US); Subhasish PURKAYASTHA, Framingham, MA (US)

(21) Appl. No.: 18/562,090

(22) PCT Filed: May 18, 2022
(86) PCT No.: PCT/IB2022/054629

§ 371 Date: Nov. 17, 2023

Related U.S. Application Data
(60) Provisional application No. 63/192,169, filed on May 24, 2021.

(51) Int. Cl. G01N 33/543; G01N 33/94

(52) U.S. Cl. CPC G01N 33/54326; G01N 33/54353; G01N 33/94

(57) ABSTRACT

Methods and kits for preparing liquid samples are presently claimed and described. The method may include treating a liquid sample with enzyme-conjugated magnetic beads are suspended in a buffer solution that comprises at least one internal standard, hydrolyzing the liquid sample to prepare a hydrolysate, and purifying the hydrolysate with magnetic based purification. Kits for preparing a liquid sample can include the, a liquid chromatography column, one or more solvents to be used as mobile phases, one or more calibrant solutions, and instructions for use.

### RELATED APPLICATIONS

The present patent application claims the priority benefit of U.S. Provisional Patent Application Ser. No. 63/192,169, filed May 24, 2021, the content of which is hereby incorporated by reference in its entirety into this disclosure.

### BACKGROUND

Liquid samples, including biological materials and bodily fluids, can often provide critical analytical information. However, many of these liquid samples include a variety of proteins that may hinder downstream analysis of small molecules. Additionally, target analytes may undergo metabolism, including glucuronidation, resulting in complexes that may prove challenging to analyze directly. To achieve a more accurate analysis of liquid samples, proper sample preparation is critical. Typical sample preparation methods may require significant time and specific conditions and are not easily automated.

### SUMMARY

The inventors have recognized the need for a sample preparation method that efficiently extracts protein from liquid samples before downstream analysis. In particular, a sample preparation method that employs both hydrolysis and magnetic bead purification, with a sample reagent comprising enzyme-conjugated magnetic beads or magnetic particles suspended in a buffer solution containing at least one internal standard. In some aspects, the buffer consists of chemically bonded hydrolysis enzyme to the surface of the magnetic bead or magnetic particle. These constituents are suspended in a buffer solution containing internal standards (isotopically labeled, non-glucuronidated, versions of the analyte) that are pertinent to a particular assay.

A magnetic bead or magnetic particle with a hydrolysis enzyme attached to it allows for convenient hydrolysis of analytes of interest. The enzyme, a protein, must be removed from hydrolysis reactions before analyzing the reaction medium with analytical equipment. The hydrolysis enzyme can cause interferences and/or equipment malfunctions if left in the reaction medium to be analyzed. An enzyme that is bound to the magnetic bead or magnetic particle surface will not enter the reaction medium and will not be subjected to contaimination or fouling of analytical equipment.

Analytical quantifications, including drug quantifications, generally rely on internal standards to correct for any source of variation. The enzyme-conjugated magnetic beads are required to be in a buffer solution for optimal function. The present disclosure adds internal standards to the buffer at specified concentrations to make a complete reagent consisting of hydrolysis enzyme and internal standard(s). During the intended use, the magnetic bead can be magnetized to a solid surface, leaving behind only supernatant consisting of the hydrolyzed analyte of interest and internal standards, resulting in a hydrolysate suspension suitable for screening and quantitative analysis on an analytical instrument.

The sample reagent (an enzyme-conjugated magnetic beads or magnetic particles suspended in a buffer solution containing internal standards) reduces the time required for sample prep., reduces the amount of waste generated per sample preparation, and enables automation. The reagent also enables the use of new technology to perform both a screen test and a definitive test on the same sample within the system. Overall, this will significantly decrease the amount of time to analyze small sample batches, will improve a user's turnaround times and will greatly improve a user's laboratory's efficiency.

One aspect of the disclosure relates to a method of preparing a liquid sample, comprising: providing a sample reagent, the sample reagent comprising: a buffer, at least one internal standard, and magnetic beads or magnetic particles, wherein a hydrolysis enzyme is conjugated to the magnetic beads or magnetic particles; incubating the sample reagent with the liquid sample to prepare a hydrolysate suspension, and purifying the hydrolysate suspension with magnetic based purification.

In another aspect, the magnetic based purification comprises magnetically separating the magnetic beads or magnetic particles from the hydrolysis suspension to produce a supernatant

In another aspect, the liquid sample is a biological sample. In yet another aspect, the biological sample is selected from the group consisting of urine, blood, oral fluid, and plasma.

In one aspect, the sample reagent is incubated with the liquid sample for at least about 5 minutes, alternatively at least about 7 minutes, alternatively at least about 10 minutes, alternatively at least about 12 minutes, alternatively at least about 15 minutes, alternatively at least about 17 minutes, alternatively at least about 20 minutes.

In one aspect, further purification of the analyte can be performed which include further purification based on immunopurification or affinity purification. In another aspect, these further purificaitons of the analyte can comprise the use of a monoclonal antibody, a polyclonal antibody, a synthetic antibody mimic, an aptamer, an affimer, DARPins, or oligonucleotides or peptides that bind to specific targets with high affinity.

In another aspect, the magnetic bead or magnetic particle comprises streptavidin and the hydrolysis enzyme comprises biotin.

In one aspect, the at least one internal standard is assay dependent. In another aspect, the at least one internal standard is selected from the group consisting of codeine-d6, morphine-d3, hydrocodone-d6, hydromorphone-d3, oxycodone-d6, oxymorphone-d3, noroxycodone-d3, norhydrocodone-d3, 7-aminoclonazepam-d4, alpha-hydroxymidazolam-d4, lorazepam-13c2-d4, oxazepam-d5, 2-hydroxyethylflurazepam-d4, alpha-hydroxyalprazolam-d5, nordiazepam-d5, temazepam-d5, alpha-hydroxytriazolam-d4, amphetamine-d5, methamphetamine-d5, eddp-d3, fentanyl-d5, methadone-d3, norfentanyl-d5, tramadol-d4, gabapentin-d10, buprenorphine-d4, and norbuprenorphine-d3. In a further aspect, the concentration of the at least one internal standard is between about 50 ng/ml to about 4800 ng/mL.

In one aspect, the hydrolysis enzyme is capable of hydrolyzing glycosidic linkages. In another aspect, the hydrolysis enzyme is β-glucuronidase, trypsin, chymotrypsin, a protease, LysC, LysN, AspN, GluC, ArgC, pronase, pepsin, prolidase. In a further aspect, the hydrolysis enzyme is capable of hydrolyzing codeine-6-glucuronide and morphine-6-glucuronide linkages.

In one aspect, the incubating further comprises mixing, shaking, or vortexing sample reagent with the liquid sample.

In one aspect, the method further comprising aliquoting the supernatant. In another aspect, the aliquoted supernatant is separated and/or enriched using a chromatography instrument, microflow, solid phase extraction, or a trap-and-elute workflow. In one aspect, the chromatography instrument is a high performance liquid chromatography (HPLC) instrument or an ultra high performance liquid chromatography instrument (UPLC).

In another aspect, the aliquoted supernatant is acoustically injected into an open port probe and transferred to an ionization source or directly injected into an ionization source. In yet another aspect, the ionized supernatant is analyzed with an analyzer. In a further aspect, the analyzer is a mass spectrometer. In one aspect, ions of interest are selected from the ionized supernatant using differential mobility spectrometry prior to analyzing the ionized supernatant with a mass spectrometer. In another embodiment, supernatant is acoustically injected into an open port probe directly from the enzymatic reaction chamber.

In one aspect, the method is used to prepare a liquid sample for clinical analysis. In another aspect, the clinical analysis is used to screen for drugs of abuse. In a further aspect, the drugs of abuse are selected from the group consisting of amphetamines, methamphetamines, benzodiazepines, barbiturates, marijuana, cocaine, PCP, methadone, and opioids (narcotics).

One aspect of the disclosure relates to a kit for preparing a liquid sample, wherein the kit comprises: a sample reagent, the sample reagent comprising: a buffer, at least one internal standard, and enzyme-conjugated magnetic beads or magnetic particles suspended in the buffer; wherein the enzyme is selected from the group consisting of β-glucuronidase, trypsin, chymotrypsin, proteases, LysC, LysN, AspN, GluC, ArgC, pronase, pepsin, prolidase, and a biotinylated enzyme; and the enzyme-conjugated magnetic beads or magnetic particles further comprising a monoclonal antibody, a polyclonal antibody, streptavidin, or have a surface coating selected from the group consisting of dextran, sodium carboxylate, phosphate, diphosphate, polyacrylic acid, oleic acid, silica, 2-hydroxypropyl trimethylammonium chloride, polyethylenimine, diethylaminoethyl cellulose, poly(4-vinylpyridine), and sodium sulfonate, and instructions for use.

In one aspect, the kit further comprises a liquid chromatography column, one or more solvents to be used as mobile phases, and/or one or more calibrant solutions.

Other aspects and features of the present disclosure will become apparent to those ordinarily skilled in the art upon review of the following description of specific aspects of the disclosure in conjunction with the accompanying figures.

### BRIEF DESCRIPTION OF THE FIGURES

Aspects of the present disclosure will now be described, by way of example only, with reference to the attached Figures, wherein:

FIG. 1 depicts a reagent according to one aspect of the disclosure.

FIG. 2 depicts the analysis of a liquid sample (Sample 1) comprising opiates using a sample reagent comprising an opiates glucuronide internal standard according to one aspect of the disclosure.

FIG. 3 depicts the analysis of a liquid sample (Sample 2) comprising oxazepam using a sample reagent comprising an oxazepam glucuronide internal standard according to one aspect of the disclosure.

FIG. 4 depicts the LC-MS/MS analysis of the liquid sample (Sample 1) depicted in FIG. 2. FIG. 5 depicts the LC-MS/MS analysis of the liquid sample (Sample 2) depicted in FIG. 3

### DETAILED DESCRIPTION

It is to be understood that this disclosure is not limited to the particular methodology, protocols, and reagents described herein and as such may vary. It is also to be understood that the terminology used herein is for the purpose of describing particular aspects only and is not intended to limit the scope of the present disclosure or the appended claims.

As used herein and in the appended claims, the singular forms “a,” “an,” and “the” include plural reference unless the context clearly indicates otherwise.

The term “about” is used in connection with a numerical value throughout the specification and the claims denote an interval of accuracy, familiar and acceptable to a person skilled in the art. In general, such an interval of accuracy is +/−10%.

Unless defined otherwise, all technical and scientific terms used herein have the same meaning as commonly understood to one of ordinary skill in the art to which this disclosure belongs.

Aspects of the disclosure include methods of preparing a liquid sample. The liquid sample may be a biological sample. Biological samples may be biological fluids, which may include, but are not limited to, blood, plasma, serum, oral fluid, or other bodily fluids or excretions, such as but not limited to saliva, urine, cerebrospinal fluid, lacrimal fluid, perspiration, gastrointestinal fluid, amniotic fluid, mucosal fluid, pleural fluid, sebaceous oil, exhaled breath, and the like.

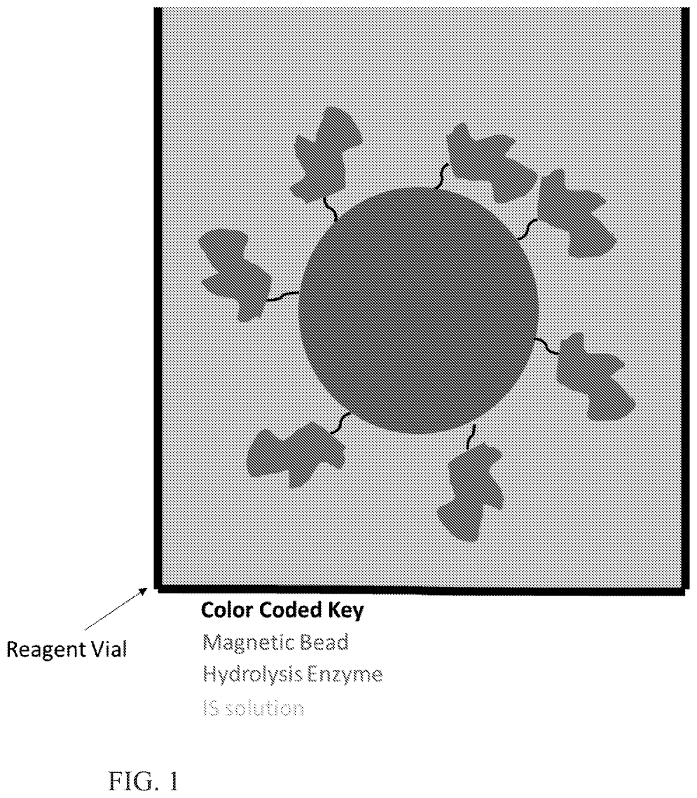

In an aspect, the method of preparing a liquid sample includes hydrolyzing the liquid samples. In this aspect, the liquid sample may be placed into a sample reagent—enzyme-conjugated magnetic beads or magnetic particles suspended in a buffer solution containing at least one internal standard. It can be appreciated by one of ordinary skill that other assay-dependent reagents may be added to the buffer solution. In an aspect, the hydrolysis enzyme is chemically bonded to the magnetic bead or magnetic particle. It can also be appreciated by one of ordinary skill that additional internal standards may be added after hydrolysis is completed. FIG. 1 depicts the enzyme-conjugated magnetic beads or magnetic particles suspended in a buffer solution containing at least one internal standard.

The sample reagent of the present disclosure will decrease the overall number of steps required for sample preparation of the liquid sample, allow one sample preparation to be used for both qualitative screening assays and subsequent quantitative definitive assays, and will increase sensitivity because the sample preparation dilution factor will be lower with the addition of only one reagent instead of many reagents.

An internal standard is a chemical substance, typically a structural analog or stable isotope of an analyte of interest. In an aspect, the internal standard may be a carbon-13 and deuterium isotopic form of the analyte of interest. Internal standards are commonly used in analytical methods to correct for variability in sample processing and analysis. The selection of internal standards is assay-dependent, and the specific internal standards selected should have similar physicochemical properties to the analyte of interest so that it behaves similarly to the analyte and reflects any changes to the analyte measurement that may occur during sample processing and analysis.

Magnetic beads or magnetic particles are typically nanoparticles or microparticles that have paramagnetic properties. Magnetic beads or magnetic particles are typically hydrophilic and disperse easily in aqueous solutions. The surface coating and/or chemistry of the magnetic beads or magnetic particles allow various biomolecules such as proteins, peptides, and nucleic acids to bind to the magnetic beads or magnetic particles. Once a biomolecule of interest is bound to a magnetic bead or magnetic particle, magnetic separation is employed to the magnetic beads or magnetic particles from a suspension by applying a magnetic force.

Enzymatic hydrolysis is a process where peptide bonds in proteins are hydrolyzed using enzymes, such as proteases, peptidases, or peptide hydrolases. Proteases can be either exopeptidases, which act near the end of a polypeptide chain and include, for example, aminopeptidases and dipeptidyl peptidases, or endopeptidases, which act on nonterminal peptide bonds and include, for example, serine proteases, cysteine proteases, aspartic acid proteases, and metallo endopeptidases. Enzymatic hydrolysis can also include the use of the glycosidase family of enzymes that catalyze the breakdown of complex carboyhydrates. For example, β-glucuronidase can catalyze the hydrolysis of β-D-glucuronic acid residues from the non-reducing end of mucopolysaccharides (also referred to as glycosaminoglycans).

Suitable hydrolysis enzymes include, but are not limited to, β-glucuronidase, trypsin, chymotrypsin, a protease, LysC, LysN, AspN, GluC, ArgC, pronase, pepsin, and prolidase. Suitable hydrolysis enzymes also include those capable of hydrolyzing glycosidic linkages, such as those formed during metabolic processes. Non-limiting examples of these glycosidic linkages include codeine-6-glucuronide and morphine-6-glucuronide linkages.

The sample reagent may be incubated with the liquid sample for a suitable amount of time. Suitable amounts of time may be dependent on the hydrolysis enzyme used and the liquid sample being hydrolyzed. Non-limiting examples of hydrolysis incubation times include about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, or about 20 minutes. In some methods, hydrolysis incubation times of greater than about 20 minutes may be employed.

The sample reagent may be incubated with the liquid sample at a suitable temperature. Suitable temperatures may be dependent on the hydrolysis enzyme used and the liquid sample being hydrolyzed. Non-limiting examples of hydrolysis temperatures include about 45° C., about 46° C., about 47° C., about 48° C., about 49° C., about 50° C., about 51° C., about 52° C., about 53° C., about 54° C., about 55° C., about 56° C., about 57° C., about 58° C., about 59 ° C., or about 60° C. In an embodiment, the sample reagent is incubated with the liquid sample for about 15 minutes at about 55° C.

In an aspect, the magnetic based purification employs the streptavidin-biotin system, where the magnetic bead or magnetic particle comprises streptavidin, and the hydrolysis enzyme comprises biotin. In an aspect embodiment, the liquid sample is hydrolyzed using the biotinylated enzyme. Subjecting the liquid sample to hydrolysis in the presence of the immobilized hydrolysis enzyme generates a hydrolysate suspension that can be acoustically ejected in an open port probe and transferred to an ionization source or directly injected into an ionization source.

In some aspects, the purifying of the hydrolysate suspension with magnetic based purification can include an optionally additional step of further purification of certain classes of drug compounds which have been extracted for analysis. These additional purificaitons can include magnetic bead or magnetic particule purification based on immunopurification or affinity purification. In these aspects, the magnetic beads or magnetic particles may comprise a monoclonal antibody, a polyclonal antibody, a synthetic antibody mimic, an aptamer, an affimer, DARPins, or oligonucleotides or peptides that bind to specific targets with high affinity

In some aspects, a method of forming a magnetic bead or magnetic particle wherein a hydrolysis enzyme is conjugated thereto includes the step of bonding/associating an activated surface of the magnetic bead or magnetic particle with an enzyme.

In some aspects, non-bound enzyme may be added to a liquid sample milieu during the liquid sample preparation. In some aspects, the enzyme may be removed by the addition of a precipitation reagent that prevents the free enzyme from interfereing with the analysis and/or the equipment. Non-limiting examples of precipitation methods include changes in temperature, pH, and/or salt concentrations or the addition of a precipitating reagent. See, e.g., Metz, Clyde, Chemistry: Inorganic Qualitative Analysis in the Laboratory, 1980. In an aspect, the proteins in the hydrolysate are precipitated using a precipitating reagent. Non-limiting examples of precipitating reagents include zinc salts, zinc sulfate, glycols, alcohols, acids, sulfates, acids, acetonitrile. Precipitating reagents may be used alone or in combination with other precipitating reagents or other organic solvents.

In an aspect, the magnetic bead purification step also comprises magnetically separating the magnetic beads or magnetic particles from the suspension to produce a supernatant. Magnetic separation may include applying a magnetic field to the magnetic beads or magnetic particles, which will draw the magnetic beads or magnetic particles toward the side wall of a sample container. The magnetic field may be applied using a magnetic rack. This allows for the removal of a biomolecule of interest bound to the magnetic beads or magnetic particles from the suspension and results in a supernatant that can be aliquoted and analyzed.

In an aspect of the disclosure, an analyzer may be used to analyze supernatant. The term “analyzer” may include any suitable instrument capable of analyzing a sample, such as a biological sample. Examples of analyzers include chromatography instruments, mass spectrometers, immunoanalyzers, hematology analyzers, microbiology analyzers, and/or molecular biology analyzers. In some cases, the supernatant may be aliquotated prior to further processing and/or analysis.

In an aspect, the supernatant is separated and/or enriched using a chromatography instrument, microflow, or solid phase extraction. Examples of chromatography instruments include, but are not limited to, a liquid chromatography instrument such as a high-performance liquid chromatography (HPLC) instrument or an ultra-high performance chromatography (UHPLC) instrument.

In an aspect, the supernatant is analyzed using a mass spectrometer. The supernatant may be introduced into the mass spectrometer with additional separation or without additional separation. If separation is not required, the supernatant may be acoustically injected into an open port probe and transferred to an ionization source or directly injected into an ionization source. Separation and/or enrichment done prior to mass spectrometry analysis may include, but is not limited to, liquid chromatography, microflow, and solid phase extraction. In some aspects, a trap-and-elute workflow may be used.

In an aspect, ions of interest are selected from the ionized supernatant using differential mobility spectrometry prior to analyzing the ionized supernatant with a mass spectrometer. Differential mobility spectrometry enables the separation of coeluting compounds, isobaric compounds, isomeric compounds, constitutional isomers, or diastereomers.

In an aspect, the method is used to prepare a liquid sample for clinical analysis. The clinical analysis can be used to screen for drugs of abuse. Non-limiting examples of drugs of abuse include amphetamines, methamphetamines, benzodiazepines, barbiturates, marijuana, cocaine, PCP, methadone, and opioids (narcotics). In an aspect, the clinical analysis is a clinical urine test or a urinalysis, and is the analysis is used to screen for drugs of abuse. Urine is a common biological sample used in testing for drugs of abuse. A urinalysis or clinical urine test can detect the presence of a drug of abuse after the drug effects have worn off. In this aspect, at least one of the following internal standards may be added to the buffer: codeine-d6, morphine-d3, hydrocodone-d6, hydromorphone-d3, oxycodone-d6, oxymorphone-d3, noroxycodone-d3, norhydrocodone-d3, 7-aminoclonazepam-d4, alpha-hydroxymidazolam-d4, lorazepam-13c2-d4, oxazepam-d5, 2-hydroxyethylflurazepam-d4, alpha-hydroxyalprazolam-d5, nordiazepam-d5, temazepam-d5, alpha-hydroxytriazolam-d4, amphetamine-d5, methamphetamine-d5, eddp-d3, fentanyl-d5, methadone-d3, norfentanyl-d5, tramadol-d4, gabapentin-d10, buprenorphine-d4, or norbuprenorphine-d3. The concentration of internal standard added is assay dependent, but may range from about 50 ng/ml to about 4800 ng/mL.

An exemplary disclosure of internal standards and working concentrations is found in Table 1.

TABLE 1

| Internal Standard | Working Concentration (ng/ml) |
|---|---|
| Codeine-d6 | 300 |
| Morphine-d3 | 600 |
| Hydrocodone-d6 | 150 |
| Hydromorphone-d3 | 250 |
| Oxycodone-d6 | 150 |
| Oxymorphone-d3 | 100 |
| Noroxycodone-d3 | 150 |
| Norhydrocodone-d3 | 150 |
| 7-Aminoclonazepam-d4 | 150 |
| Alpha-hydroxymidazolam-d4 | 100 |
| Lorazepam-13C2-d4 | 100 |
| Oxazepam-d5 | 50 |
| 2-Hydroxyethylflurazepam-d4 | 450 |
| Alpha-hydroxyalprazolam-d5 | 150 |
| Nordiazepam-d5 | 200 |
| Temazepam-d5 | 300 |
| Alpha-hydroxytriazolam-d4 | 600 |
| Amphetamine-d5 | 500 |
| Methamphetamine-d5 | 500 |
| EDDP-d3 | 500 |
| Fentanyl-d5 | 500 |
| Methadone-d3 | 500 |
| Norfentanyl-d5 | 500 |
| Tramadol-d4 | 500 |
| Gabapentin-d10 | 4800 |
| Buprenorphine-d4 | 500 |
| Norbuprenorphine-d3 | 500 |

One aspect of the disclosure includes a kit for preparing a liquid sample for analysis. The kit comprises instructions for use and a sample reagent, the sample reagent comprising: a buffer, at least one internal standard, enzyme-conjugated magnetic beads or magnetic particles suspended in the buffer wherein the enzyme-conjugated magnetic beads or magnetic particles further comprise a monoclonal antibody, a polyclonal antibody, streptavidin, or have a surface coating selected from the group consisting of dextran, sodium carboxylate, phosphate, diphosphate, polyacrylic acid, oleic acid, silica, 2-hydroxypropyl trimethylammonium chloride, polyethylenimine, diethylaminoethyl cellulose, poly(4-vinylpyridine), and sodium sulfonate. Non-limiting examples of the enzyme include β-glucuronidase, trypsin, chymotrypsin, a protease, LysC, LysN, AspN, GluC, ArgC, pronase, pepsin, prolidase, or a biotinylated enzyme.

The kit may also include a liquid chromatography column. The liquid chromatography column may be a reversed-phase chromatography column. Non-limiting examples of reversed-phase chromatography columns include C18-bonded silica, C8-bonded silica, pure silica, cyano-bonded silica, and phenyl-bonded silica. The kit may also include one or more solvents. Non-limiting examples of suitable chromatography solvents include organic solvents, such as acetonitrile, methanol, and propanol. To improve the chromatographic peak shape, acids, such as formic acid, trifluoroacetic acid, or acetic acid, may be included. These acids also provide a source of protons in reversed-phase LC/MS applications. The kit may also include one or more calibrant or calibration solution. The calibrant or calibration solution may be used to calibrate an analyzer. The kit may also include instructions for use.

### EXAMPLES

### Example 1: Exemplary Sample Preparation and Analysis Method

Urine samples can be collected according to the SAMHSA guidelines (Substance Abuse and Mental Health Services Administration Center for Substance Abuse Prevention), available at https://www.samhsa.gov/sites/default/files/specimen-collection-handbook-2014.pdf. Urine specimens are typically submitted to certified laboratories within 24 hours after collection.

A 50 μL aliquot of a urine sample and 20 μL of the sample reagent according to the aspects of the disclosure are added to a plastic reaction vessel. The urine sample is mixed via vortexing and is subsequently hydrolyzed by a fast-acting enzyme that is proven capable of hydrolyzing glycosidic linkages in 15 minutes at 55° C. After vortexing briefly, a magnetic rack is used to pull the magnetic beads with the to one side of the reaction vessel, leaving behind a very clear supernatant that contains the analyte(s) of interest. This supernatant can then be easily aliquoted into an HPLC vial or directly injected into a mass spectrometer via direct flow injection, a trap and elute system, and an open port probe.

Example 2: Analysis of Drugs of Abuse

A urine sample was prepared using the procedures of Example 1. 50 μL of the resulting urine sample was transferred to an Echo qualified microplate. Using an open port probe, 2.5 nL of the prepared urine samples were introduced into an Echo MS system. After Echo MS analysis, an aliquot from the Echo qualified microplate was diluted 12× with water, placed in an HPLC vial, and was anlayzed with microflow LC-MS/MS definitive testing.

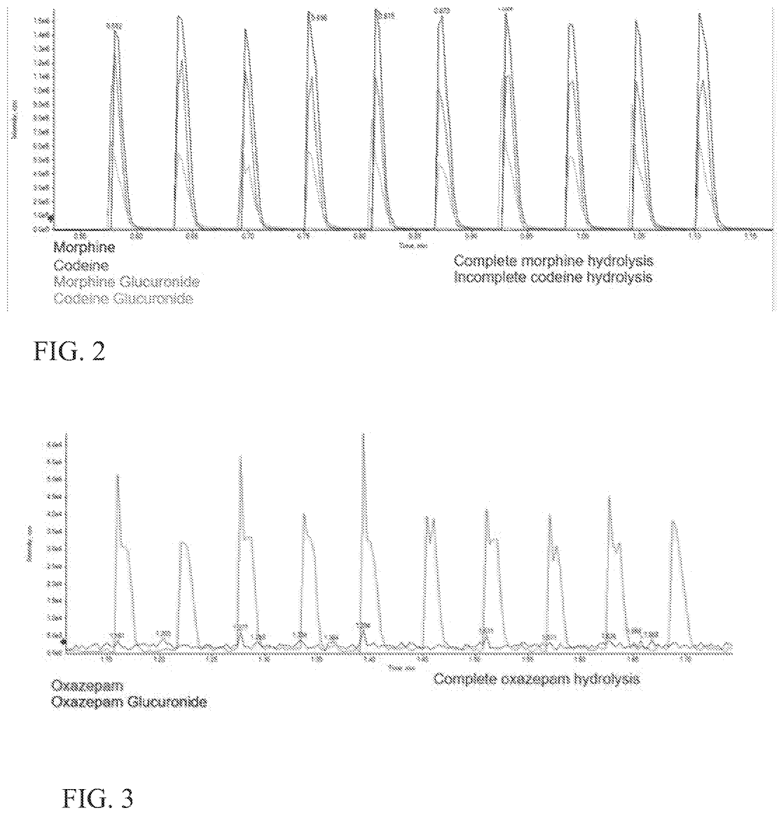

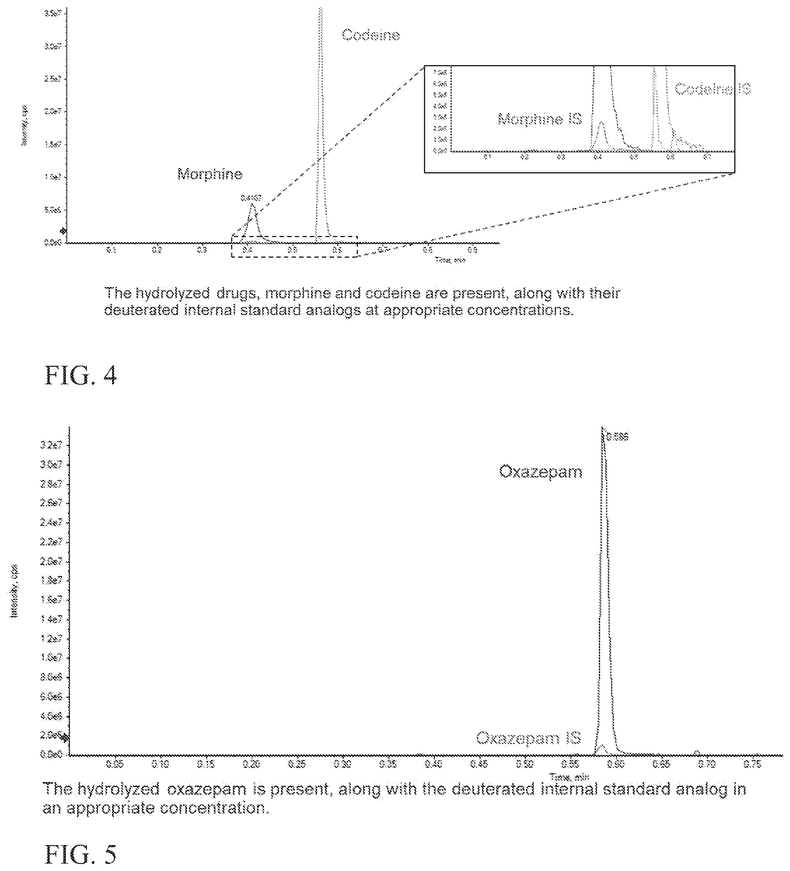

Two different samples were run using this method. Sample 1 consisted of urine samples prepared with a sample reagent comprising the internal standards morphine glucuronide and codeine glucuronide. Sample 2 consisted of urine samples prepared with a sample reagent comprising the internal standard oxazepam glucuronide. Sample 1 and Sample 2 were independently analyzed ten (10) consecutive times, and the resulting signal intensities are depicted in FIG. 2 (Sample 1) and FIG. 3 (Sample 2). Definitive testing of Samples 1 and 2 was carried out using an M5 Microflow LC-MS/MS instrument. The resulting spectrograms are depicted in FIG. 4 (Sample 1) and FIG. 5 (Sample 2). Definitive testing could also be carried out using DMS-MS/MS.

While the present disclosure has been described with reference to certain aspects, it will be understood by those skilled in the art that various changes may be made and equivalents may be substituted without departing from the scope of the present disclosure or appended claims. In addition, many modifications may be made to adapt a particular situation or material to the teachings of the present disclosure without departing from its scope. Therefore, it is intended that the present disclosure not be limited to the particular aspects disclosed, but that the present disclosure will include all aspects falling within the scope of the appended claims.

All patents, patent applications, publications, and descriptions mentioned above are herein incorporated by reference in their entirety.

1. A method of preparing a liquid sample, comprising:
providing a sample reagent, the sample reagent comprising
a buffer,
at least one internal standard,
and magnetic beads or magnetic particles, wherein a hydrolysis enzyme is conjugated to the magnetic beads or magnetic particles,
incubating the sample reagent with a liquid sample to prepare a hydrolysate suspension,
and
purifying the hydrolysate suspension with magnetic based purification.

2. The method of claim 1, wherein the magnetic based purification comprises magnetically separating the magnetic beads or magnetic particles from the hydrolysate suspension to produce a supernatant.

3. The method of claim 1, wherein the liquid sample comprises a biological sample selected from the group consisting of urine, blood, oral fluid, and plasma.

4. (canceled)

5. The method of claim 1, wherein the sample reagent is incubated with the liquid sample for at least about 5 minutes.

6. The method of claim 1, wherein the magnetic beads or magnetic particles comprise streptavidin and the hydrolysis enzyme comprises biotin.

7. The method of claim 1, wherein the at least one internal standard is assay dependent.

8. The method of claim 1, wherein the at least one internal standard is selected from the group consisting of codeine-d6, morphine-d3, hydrocodone-d6, hydromorphone-d3, oxycodone-d6, oxymorphone-d3, noroxycodone-d3, norhydrocodone-d3, 7-aminoclonazepam-d4, alpha-hydroxymidazolam-d4, lorazepam-13c2-d4, oxazepam-d5, 2-hydroxyethylflurazepam-d4, alpha-hydroxyalprazolam-d5, nordiazepam-d5, temazepam-d5, alpha-hydroxytriazolam-d4, amphetamine-d5, methamphetamine-d5, eddp-d3, fentanyl-d5, methadone-d3, norfentanyl-d5, tramadol-d4, gabapentin-d10, buprenorphine-d4, and norbuprenorphine-d3.

9. The method of claim 1, wherein the concentration of the at least one internal standard is between about 50 ng/ml to about 4800 ng/mL.

10. The method of claim 1, wherein the hydrolysis enzyme is capable of hydrolyzing glycosidic linkages.

11. The method of claim 1, wherein the hydrolysis enzyme is β-glucuronidase, trypsin, chymotrypsin, a protease, LysC, LysN, AspN, GluC, ArgC, pronase, pepsin, prolidase.

12. The method of claim 1, wherein the hydrolysis enzyme is capable of hydrolyzing codeine-6-glucuronide and morphine-6-glucuronide linkages.

13. (canceled)

14. The method of claim 2, further comprising aliquoting the supernatant, wherein the aliquoted supernatant is separated and/or enriched using a chromatography instrument, microflow, solid phase extraction, or a trap-and-elute workflow.

15. (canceled)

16. (canceled)

17. The method of claim 14, further comprising acoustically injecting into an open port probe and transferring to an ionization source or directly injected into an ionization source the aliquoted supernatant.

18. The method of claim 17, wherein the ionized supernatant is analyzed with a mass spectrometer an analyzer.

19. (canceled)

20. The method of claim 18, further comprising selecting ions of interest from the ionized supernatant using differential mobility spectrometry.

21. The method of claim 1, wherein the method is used to prepare a liquid sample for clinical analysis.

22. The method of claim 21, wherein the clinical analysis is used to screen for drugs of abuse.

23. The method of claim 22, wherein the drugs of abuse are selected from the group consisting of amphetamines, methamphetamines, benzodiazepines, barbiturates, marijuana, cocaine, PCP, methadone, and opioids (narcotics).

24. A kit for preparing a liquid sample, wherein the kit comprises:
a sample reagent comprising:
a buffer,
at least one internal standard, and
enzyme-conjugated magnetic beads or magnetic particles suspended in the buffer;
wherein the enzyme is selected from the group consisting of β-glucuronidase, trypsin, chymotrypsin, proteases, LysC, LysN, AspN, GluC, ArgC, pronase, pepsin, prolidase, and a biotinylated enzyme,
and instructions for use.

25. The kit of claim 24, wherein the kit further comprises a liquid chromatography column, one or more solvents to be used as mobile phases, and/or one or more calibrant solutions.
